(19) United States
(12) Patent Application Publication
(10) Pub. No.: US 2005/0214841 A1
(43) Pub. Date: Sep. 29, 2005

(54) Substrate for biochip

(76) Inventor: Koichiro Nakamura, Tokyo (JP)

Correspondence Address: WHITHAM, CURTIS & CHRISTOFFERSON, P.C., 11491 SUNSET HILLS ROAD, SUITE 340, RESTON, VA, 20190, US

(21) Appl. No.: 11/087,616

(22) Filed: Mar. 24, 2005

(30) Foreign Application Priority Data
Mar. 25, 2004 (JP) P2004-088406

(51) Int. Cl. C12Q 1/68; C12M 1/34

(52) U.S. Cl. 435/6; 435/287.2

(57) ABSTRACT

A substrate having a plurality of recesses, wherein each of the plurality of recesses has a surface, wherein at least part of the surface is coated with a metal film comprising at least one element selected from Au, Ag, Cu and Pd. A biochip substrate comprising: a substrate having at least one recess; and a metal film formed on the at least one recess, wherein the metal film comprises at least one element selected from Au, Ag, Cu and Pd.

### BACKGROUND OF THE INVENTION

1. Field of the Invention

The present invention relates to a biochip to be used in the field of bioscience and the like, and in particular relates to a substrate for a biochip which has a function of selectively attaching or retaining a specific substance in a small area.

2. Description of the Related Art

In the field of bioscience, the development of higher-integrated functional elements and higher-density arrays has been made for ultratrace analysis or ultrasensitive analysis by using a microchemical reactor, a chip for genomic analysis, a chip for protein analysis or the like. Accordingly, for the substrates to be used for these analyses, selective adhesiveness has been required. Such a substrate can selectively retain a small amount of a liquid sample such as a solution of a biological substance in a specified site and can provide the sample for analysis or reaction.

Such a function can be attained by forming sites having a function of binding a molecule of a specific substance (functional binding site) in a high density on the surface of a substrate. Such a technique has been disclosed in, for example, JP-T-9-500568, JP-A-2002-131327, JP-A-2002-307801, JP-A-2002-283530, JP-A-2003-121442 and the like.

### SUMMARY OF THE INVENTION

However, all the methods disclosed in the foregoing JP-T-9-500568, JP-A-2002-131327, JP-A-2002-307801, JP-A-2002-283530 and JP-A-2003-121442 are a method of forming a pattern on the flat surface of a substrate. Since a functional binding site is present in the flat portion, there were problems that the retained amount largely varies when small amounts of a sample such as a biological substance are retained in plural sites on the surface of the substrate, and that the repetitive reproducibility is bad. In addition, when the binding sites are densified, adjacent binding sites get closer to each other, therefore there was a problem that contamination of an adjacent sample occurs.

The present invention has been conducted in order to solve the foregoing problems, and an object of the invention is to provide a substrate for a biochip which can attach or retain a small amount of a specific substance in a small area in a high density with a good reproducibility.

To solve the foregoing problems, the invention provides the following:

(1) A substrate having a plurality of recesses,
wherein each of the plurality of recesses has a surface,
wherein at least part of the surface is coated with a metal film comprising at least one element selected from Au, Ag, Cu and Pd.

(2) The substrate as described in (1) above,
wherein the plurality of recesses are regularly arranged.

(3) The substrate as described in (1) or (2) above,
wherein a linker for immobilizing a biological substance is bound to the metal film.

(4) The substrate as described in (3) above,
wherein the liker has a thioether bond bound to the metal film.

(5) The substrate as described in any of (1) to (4) above,
wherein a surface of the substrate other than the at least part of the surface is coated with a water-repellent film.

(6) A biochip substrate comprising:
a substrate having at least one recess; and
a metal film formed on the at least one recess,
wherein the metal film comprises at least one element selected from Au, Ag, Cu and Pd.

(7) The biochip substrate as described in (6) above,
wherein the metal film covers the at least one recess entirely.

(8) The biochip substrate as described in (6) above,
wherein the metal film is coated on a bottom portion of the at least one recess.

(9) The biochip substrate as described in any of (6) to (8) above,
wherein the at least one recess is regularly arranged.

(10) The biochip substrate as described in any of (6) to (9) above, which further comprises a linker for immobilizing a biological substance,
wherein the linker is bound to the metal film.

(11) The biochip substrate as described in (10) above,
wherein the liker has a thioether bond bound to the metal film.

(12) The biochip substrate as described in any of (6) to (11) above, which further comprises a water-repellent film covering a surface of the biochip other than a surface of the metal film.

(13) The biochip substrate as described in (12) above, wherein the water-repellent film further covers a part of a surface of the at least one recess.

In the recess coated with the metal film described above, a specific chemical substance having an affinity for such a metal can be attached or retained in a small area with a good reproducibility.

By binding, to the recess of the substrate, a linker having an affinity for the foregoing metal and having a functional group with a function of selectively immobilizing a biological substance, a biological substance such as DNA can be effectively attached or retained in a small area.

Further, a specific chemical substance is attached only to the specified portion and will be difficult to attach to the portion other than the specified portion, whereby the selectivity can be enhanced.

### BRIEF DESCRIPTION OF THE DRAWINGS

FIG. 1 is a perspective view showing an example of a substrate for a biochip of the present invention;

FIG. 2 is a cross-sectional schematic view of an example of a substrate for a biochip;

FIG. 3 is a view illustrating a contact angle of a liquid droplet;

FIG. 4 shows diagrams illustrating processes for modifying a recess of a substrate; and

FIG. 5 shows diagrams illustrating processes for binding DNA.

### DETAILED DESCRIPTION OF THE INVENTION

Hereunder, embodiments of the present invention will be described in detail.

An example of a substrate for a biochip of the present invention is shown in FIG. 1. On the surface of a substrate 10 in the shape of a flat plate, plural recesses 20 for retaining a liquid material such as a solution of a biological substance are formed. In this example, a flat portion 30, which is the surface of the original substrate in the shape of a flat plate, is present between adjacent recesses. By performing a treatment so as to impart a difference in adhesiveness to a specific sample of a biological substance between the surface of the recesses and the surface of the flat portion of the substrate other than the recesses, the ability of retaining the sample in the recesses 20 can be improved.

Examples of a material to be used for the substrate of the present invention can include a glass, ceramics, semiconductor, metal, resin and the like. As the types of the glass that can be utilized, silica glass (linear expansion coefficient: α=0.5 ppm/K), non-alkali glass, soda lime glass and the like can be exemplified. Further, a low expansion crystallized glass such as Zerodur (Schott Inc., α=−2 ppm/K) and Neoceram (Nippon Electric Glass Co., Ltd., α=0.15 ppm/K), Pyrex (Corning Co., Ltd., α=3.25 ppm/K), BK7 (Schott Inc., α=7.1 ppm/K) and the like can be exemplified.

In addition, a semiconductor material such as silicon in a wafer form, InP or GaAs can be also used. As a resin material, an epoxy resin, acrylic resin, polycarbonate resin, polyimide resin, fluororesin and the like can be exemplified. Among these, it is most preferred to use glass which is excellent in heat resistance, transparency and chemical stability.

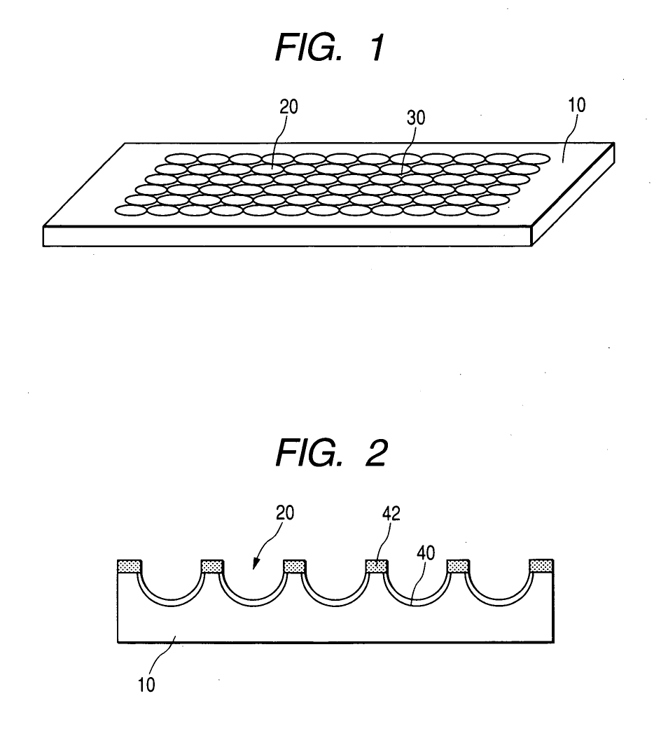

FIG. 2 shows a sectional view of a substrate for a biochip of the present invention. A metal film 40 is formed on the surface in the recesses 20 provided on the substrate 10 in the shape of a flat plate, and a water-repellent film 42 is formed on the surface of the flat portion. A typical metal film is a gold (Au) film, however, it is not limited thereto, and silver (Ag), copper (Cu), palladium (Pd) and the like can be also used.

In FIG. 2, a metal film is formed on the entire surface in the recess 20, however, it may be formed on a specified portion, for example, only a bottom portion of the recess as needed.

Further, a linker having a functional group with a function of selectively immobilizing a biological substance and a compound that binds to such a biological substance is introduced on the surface of the metal film described above.

The biological substance herein refers to a nucleic acid such as DNA or RNA, a protein, lipid, saccharide, vitamin, hormone, enzyme or the like.

Examples of the functional group that can selectively immobilize such a biological substance can include an amino group, mercapto group, carboxyl group, sulfonic acid group, hydroxyl group, alkyl group, phenyl group and the like.

Among these, it is preferred to use a compound having a mercapto group that has a high affinity for Au, Ag, Cu or Pd, and a carboxyl group that can chemically bind a biological substance. As such a compound, 3-mercaptopropionic acid and 3,3′-dithiodipropionic acid are preferred.

Other than these, an alkyl thiol compound, hydroxyalkyl thiol compound or aminoalkylthiol compound, which contains an alkyl group, hydroxyl group, amino group or the like may be used. In addition, an alkyl disulfide compound, alkyl disulfide compound containing a hydroxyl group, alkyl disulfide compound containing a carboxyl group and alkyl disulfide compound containing an amino group, which are disulfide compounds thereof can be exemplified.

Further, a lipid (thiolipid) that has a SH group in one terminal and a dialkyl group in the other terminal may be bound to the Au film in the recess via Au—S bond.

Alternatively, a bilayer that is constituted by mixing abovementioned thiolipid with phospholipids such as di-oleoyl phosphatidyl choline (produced by SIGMA-ALDRICH, Inc.) and di-phytanoyl phosphatidyl choline may be bound to the Au film in the recess via Au—S bond between thiolipid and Au film.

Additionally, abovementioned bilayer may be a membrane protein that comprises a protein.

Specific examples thereof can include alkanethiols such as CH₃(CH₂)₃₀SH, CH₃(CH₂)₂₅SH, CH₃(CH₂)₂₀SH, CH₃(CH₂)₁₉SH, CH₃(CH₂)₁₈SH, CH₃(CH₂)₁₇SH, CH₃(CH₂)₁₆SH, CH₃(CH₂)₁₅SH, CH₃(CH₂)₁₄SH, CH₃(CH₂)₁₃SH, CH₃(CH₂)₁₂SH, CH₃(CH₂)₁₁SH, CH₃(CH₂)₁₀SH, CH₃(CH₂)₉SH, CH₃(CH₂)₈SH, CH₃(CH₂)₇SH, CH₃(CH₂)₆SH, CH₃(CH₂)₅SH, CH₃(CH₂)₄SH, CH₃(CH₂)₃SH, CH₃(CH₂)₂SH, and CH₃CH₂SH, alkanethiols containing a hydroxyl group such as HOCH₂(CH₂)₃₀SH, HOCH₂(CH₂)₂₅SH, HOCH₂(CH₂)₂₀SH, HOCH₂(CH₂)₁₉SH, HOCH₂(CH₂)₁₈SH, HOCH₂(CH₂)₁₇SH, HOCH₂(CH₂)₁₆SH, HOCH₂(CH₂)₁₅SH, HOCH₂(CH₂)₁₄SH, HOCH₂(CH₂)₁₃SH, HOCH₂(CH₂)₁₂SH, HOCH₂(CH₂)₁₁SH, HOCH₂(CH₂)₁₀SH, HOCH₂(CH₂)₉SH, HOCH₂(CH₂)₇SH, HOCH₂(CH₂)₆SH, HOCH₂(CH₂)₅SH, HOCH₂(CH₂)₄SH, HOCH₂(CH₂)₃SH, HOCH₂(CH₂)₂SH, and HOCH₂CH₂SH, alkanethiols containing a carboxyl group such as HOOC(CH₂)₃₀SH, HOOC(CH₂)₂₅SH, HOOC(CH₂)₂₀SH, HOOC(CH₂)₁₉SH, HOOC(CH₂)₁₈SH, HOOC(CH₂)₁₇SH, HOOC(CH₂)₁₆SH, HOOC(CH₂)₁₅SH, HOOC(CH₂)₁₄SH, HOOC(CH₂)₁₃SH, HOOC(CH₂)₁₂SH, HOOC(CH₂)₁₁SH, HOOC(CH₂)₁₀SH, HOOC(CH₂)₉SH, HOOC (CH₂)₈SH, HOOC(CH₂)₇SH, HOOC(CH₂)₆SH, HOOC(CH₂)₅SH, HOOC(CH₂)₄SH, HOOC(CH₂)₃SH, HOOC(CH₂)₂SH, and HOOCCH₂SH, alkanethiols containing an amino group such as H₂N(CH₂)₃₀SH, H₂N(CH₂)₂₅SH, H₂N(CH₂)₂₀SH, H₂N(CH₂)₁₉SH, H₂N(CH₂)₁₈SH, H₂N(CH₂)₁₇SH, H₂N(CH₂)₁₆SH, H₂N(CH₂)₁₅SH, H₂N(CH₂)₁₄SH, H₂N(CH₂)₁₃SH, H₂N(CH₂)₁₂SH, H₂N(CH₂)₁₁SH, H₂N(CH₂)₁₀SH, H₂N(CH₂)₉SH, H₂N(CH₂)₈SH, H₂N(CH₂)₇SH, H₂N(CH₂)₆SH, H₂N(CH₂)₅SH, H₂N(CH₂)₄SH, H₂N(CH₂)₃SH, H₂N(CH₂)₂SH, and H₂NCH₂SH, alkyl disulfide compounds such as [CH₃(CH₂)₃₀S]₂, [CH₃(CH₂)₂₅S]₂, [CH₃(CH₂)₂₀S]₂, [CH₃(CH₂)₁₉S]₂, [CH₃(CH₂)₁₈S]₂, [CH₃(CH₂)₁₇S]₂, [CH₃(CH₂)₁₆S]₂, [CH₃(CH₂)₁₅S]₂, [CH₃(CH₂)₁₄S ]2, [CH₃(CH₂)₁₃S]₂, [CH₃(CH₂)₁₂S]₂, [CH₃(CH₂)₁₁S]₂, [CH₃(CH₂)₁₀S]₂, [CH₃(CH₂)₉S]₂, [CH₃(CH₂)₈S ]₂, [CH₃(CH₂)₇S)₂, [CH₃(CH₂)₆S]₂, [CH₃(CH₂)₅S]₂, [CH₃(CH₂)₄S ] 2, [CH₃(CH₂)₃S]₂, [CH₃(CH₂)₂S]₂, and [CH₃CH₂S]₂, alkyl disulfide compounds containing a hydroxyl group such as [HOCH₂(CH₂)₃₀S]₂, [HOCH₂(CH₂)₂₅S]₂, [HOCH₂(CH₂)₂₀S]₂, (HOCH₂(CH₂)₁₉S]₂, [HOCH₂(CH₂)₁₈S]₂, [HOCH₂(CH₂)₁₇S]₂, [HOCH₂(CH₂)₁₆S]₂, [HOCH₂(CH₂)₁₅S]₂, [HOCH₂(CH₂)₁₄S]₂, [HOCH₂(CH₂)₁₃S]₂, [HOCH₂(CH₂)₁₂S]₂, [HOCH₂(CH₂)₁₁S]₂, [HOCH₂(CH₂)₁₀S]₂, [HOCH₂(CH₂)₉S]₂, [HOCH₂(CH₂)₈S]₂, [HOCH₂(CH₂)₇S ]₂, [HOCH₂(CH₂)₆S]₂, [HOCH₂(CH₂)₅S]₂, [HOCH₂(CH₂)₄S]₂, [HOCH₂(CH₂)₃S]₂, [HOCH₂(CH₂)₂S]₂, and [HOCH₂CH₂S]₂, alkyl disulfide compounds containing a carboxyl group such as [HOOC(CH₂)₃₀S]₂, [HOOC(CH₂)₂₅S]₂, [HOOC(CH₂)₂₀S]₂, [HOOC(CH₂)₁₉S]₂, [HOOC(CH₂)₁₈S]₂, [HOOC(CH₂)₁₇S]₂, [HOOC(CH₂)16S]₂, [HOOC(CH₂)₁₅S]₂, [HOOC(CH₂)₁₄S]₂, [HOOC(CH₂)₁₃S]₂, [HOOC(CH₂)₁₂S]₂, [HOOC(CH₂)₁₁S]₂, [HOOC (CH₂)₁₀S]₂, [HOOC (CH₂)₉S]₂, [HOOC(CH₂)₈S]₂, [HOOC(CH₂)₇S]₂, [HOOC(CH₂)₆S]₂, [HOOC(CH₂)₅S]₂, [HOOC(CH₂) ₄S]₂, [HOOC(CH₂)₃S]₂, [HOOC(CH₂)₂S]₂, and [HOOCCH₂S]₂, alkyl disulfide compounds containing an amino group such as [H₂N (CH₂)₃₀S]₂, [H₂N (CH₂)₂₅S]₂, [H₂N(CH₂)₂₀S]₂, [H₂N(CH₂)₁₉S]₂, [H₂N(CH₂)₁₈S]₂, [H₂N(CH₂)₁₇S]₂, [H₂N(CH₂)₁₆S]₂, [H₂N(CH₂)₁₅S]₂, [H₂N (CH₂)₁₄S]₂, [H₂N(CH₂)₁₃S]₂, [H₂N(CH₂)₁₂S]₂, [H₂N(CH₂)₁₁S]₂, [H₂N(CH₂)₁₀S]₂, [H₂N(CH₂)₉S]₂, [H₂N (CH₂)₈S]₂, [H₂N(CH₂)₇S]₂, [H₂N(CH₂)₆S]₂, [H₂N(CH₂)₅S]2₁, [H₂N(CH₂)₄S]₂, [H₂N(CH₂)₃S]₂, [H₂N(CH₂)₂S]₂, and [H₂NCH₂S]₂.

On the other hand, it is preferred that the portion other than the specified portion on the surface of the recess of the substrate, particularly the surface of the flat portion of the substrate is water repellent. For example, a part of the surface of the recess may be water repellent. As a material that imparts a water repellency, tetrafluoroethylene, cyclic perfluoropolymer, fluoroalkylsilane, alkylsilane, silicone, polysilane etc., which have a water-repellent group, can be exemplified.

As a compound having a water-repellent group, a silane compound having a water-repellent group is preferably used. Examples thereof can include a silane compound having one or more water-repellent groups such as an alkyl group, fluoroalkyl group and the like in the molecule.

Examples of the silane compound having an alkyl group can include chlorosilanes containing an alkyl group such as CH₃(CH₂)₃₀SiCl₃, CH₃(CH₂)₂₀SiCl₃, CH₃(CH₂)₁₈SiCl₃, CH₃(CH₂)₁₆SiCl₃, CH₃(CH₂)₁₄SiCl₃, CH₃(CH₂)₁₂SiCl₃, CH₃(CH₂)₁₀SiCl₃, CH₃(CH₂)₉SiCl₃, CH₃(CH₂)₈SiCl₃, CH₃(CH₂)₇SiCl₃, CH₃(CH₂)₆SiCl₃, CH₃(CH₂)₅SiCl₃, CH₃(CH₂)₄SiCl₃, CH₃(CH₂)₃SiCl₃, CH₃(CH₂)₂SiCl₃, CH₃CH₂SiCl₃, (CH₃CH₂)₂SiCl₂, (CH₃CH₂)₃SiCl, CH₃SiCl₃, (CH₃)₂SiCl₂ and (CH₃)₃SiCl, alkoxysilanes containing an alkyl group such as CH₃(CH₂)₃₀Si(OCH₃)₃, CH₃(CH₂)₂₀Si(OCH₃)₃, CH₃(CH₂)₁₈Si(OCH₃)₃, CH₃(CH₂)₁₆Si(OCH₃)₃, CH₃(CH₂)₁₄Si(OCH₃)₃, CH₃(CH₂)₁₂Si(OCH₃)₃, CH₃(CH₂)₁₀Si(OCH₃)₃, CH₃(CH₂)₉Si(OCH₃)₃, CH₃(CH₂)₈Si(OCH₃)₃, CH₃(CH₂)₇Si(OCH₃)₃, CH₃(CH₂)₆Si(OCH₃)₃, CH₃(CH₂)₅Si(OCH₃)₃, CH₃(CH₂)₄Si(OCH₃)₃, CH₃(CH₂)₃Si(OCH₃)₃, CH₃(CH₂)₂Si(OCH₃)₃, CH₃CH₂Si(OCH₃)₃, (CH₃CH₂)₂Si(OCH₃)₂, (CH₃CH₂)₃SiOCH₃, CH₃Si(OCH₃)₃, (CH₃)₂Si(OCH₃)₂, (CH₃)₃SiOCH₃, CH₃(CH₂)₃₀Si(OC₂H₅)₃, CH₃(CH₂)₂₀Si(OC₂H₅)₃, CH₃(CH₂)₁₈Si(OC₂H₅)₃, CH₃(CH₂)₁₆Si(OC₂H₅)₃, CH₃(CH₂)₁₄Si(OC₂H₅)₃, CH₃(CH₂)₁₂Si(OC₂H₅)₃, CH₃(CH₂)₁₀Si(OC₂H₅)₃, CH₃(CH₂)₉Si(OC₂H₅)₃, CH₃(CH₂)₈Si(OC₂H₅)₃, CH₃(CH₂)₇Si(OC₂H₅)₃, CH₃(CH₂)₆Si(OC₂H₅)₃, CH₃(CH₂)₅Si(OC₂H₅)₃, CH₃(CH₂)₄Si(OC₂H₅)₃, CH₃(CH₂)₃Si(OC₂H₅)₃, CH₃(CH₂)₂Si(OC₂H₅)₃, CH₃CH₂Si(OC₂H₅)₃, (CH₃CH₂)₂Si(OC₂H₅)₂, (CH₃CH₂)₃SiOC₂H₅, CH₃Si(OC₂H₅)₃, (CH₃)₂Si(OC₂H₅)₂ and (CH₃)₃SiOC₂H₅, acyloxysilanes containing an alkyl group such as CH₃(CH₂)₃₀Si(OCOCH₃)₃, CH₃(CH₂)₂₀Si(OCOCH₃)₃, CH₃(CH₂)₁₈Si(OCOCH₃)₃, CH₃(CH₂)₁₆Si(OCOCH₃)₃, CH₃(CH₂)₁₄Si(OCOCH₃)₃, CH₃(CH₂)₁₂Si(OCOCH₃)₃, CH₃(CH₂)₁₀Si(OCOCH₃)₃, CH₃(CH₂)₉Si(OCOCH₃)₃, CH₃(CH₂)₈Si(OCOCH₃)₃, CH₃(CH₂)₇Si(OCOCH₃)₃, CH₃(CH₂)₆Si(OCOCH₃)₃, CH₃(CH₂)₅Si(OCOCH₃)₃, CH₃(CH₂)₄Si(OCOCH₃)₃, CH₃(CH₂)₃Si(OCOCH₃)₃, CH₃(CH₂)₂Si(OCOCH₃)₃, CH₃CH₂Si(OCOCH₃)₃, (CH₃CH₂)₂Si(OCOCH₃)₂, (CH₃CH₂)₃SiOCOCH₃, CH₃Si(OCOCH₃)₃, (CH₃)₂Si(OCOCH₃)₂ and (CH₃)₃SiOCOCH₃, isocyanate silanes containing an alkyl group such as CH₃(CH₂)₃₀Si(NCO)₃, CH₃(CH₂)₂₀Si(NCO)₃, CH₃(CH₂)₁₈Si(NCO)₃, CH₃(CH₂)₁₆Si(NCO)₃, CH₃(CH₂)₁₄Si(NCO)₃, CH₃(CH₂)₁₂Si(NCO)₃, CH₃(CH₂)₁₀Si (NCO)₃, CH₃(CH₂)₉Si(NCO)₃, CH₃(CH₂)₈Si(NCO)₃, CH₃(CH₂)₇Si(NCO)₃, CH₃(CH₂)₆Si(NCO)₃, CH₃(CH₂)₅Si(NCO)₃, CH₃(CH₂)₄Si(NCO)₃, CH₃(CH₂)₃Si(NCO)₃, CH₃(CH₂)₂Si(NCO)₃, CH₃CH₂Si(NCO)₃, (CH₃CH₂)₂Si(NCO)₂, (CH₃CH₂)₃SiNCO, CH₃Si(NCO)₃, (CH₃)₂Si(NCO)₂ and (CH₃) ₃SiNCO.

Examples of the silane compound having a fluoroalkyl group can include trichlorosilanes containing a fluoroalkyl group such as CF₃(CF₂)₁₁(CH₂)₂SiCl₃, CF₃(CF₂)₁₀(CH₂)₂SiCl₃, CF₃(CF₂)₉(CH₂)₂SiCl₃, CF₃(CF₂)₈(CH₂)₂SiCl₃, CF₃(CF₂)₇(CH₂)₂SiCl₃, CF₃(CF₂)₆(CH₂)₂SiCl₃, CF₃(CF₂)₅(CH₂)₂SiCl₃, CF₃(CF₂)₄(CH₂)₂SiCl₃, CF₃(CF₂)₃(CH₂)₂SiCl₃, CF₃(CF₂)₂(CH₂)₂SiCl₃, CF₃CF₂(CH₂)₂SiCl₃ and CF₃(CH₂)₂SiCl₃, trialkoxysilanes containing a fluoroalkyl group such as CF₃(CF₂)₁₁(CH₂)₂Si(OCH₃)₃, CF₃(CF₂)₁₀(CH₂)₂Si(OCH₃)₃, CF₃(CF₂)₉(CH₂)₂Si(OCH₃)₃, CF₃(CF₂)₈(CH₂)₂Si(OCH₃)₃, CF₃(CF₂)₇(CH₂)₂Si(OCH₃)₃, CF₃(CF₂)₆(CH₂)₂Si(OCH₃)₃, CF₃(CF₂)₅(CH₂)₂Si(OCH₃)₃, CF₃(CF₂)₄(CH₂)₂Si(OCH₃)₃, CF₃(CF₂)₃(CH₂)₂Si(OCH₃)₃, CF₃(CF₂)₂(CH₂)₂Si(OCH₃)₃, CF₃CF₂(CH₂)₂Si(OCH₃)₃, CF₃(CH₂)₂Si(OCH₃)₃, CF₃(CF₂)₁₁(CH₂)₂Si(OC₂H₅)₃, CF₃(CF₂)₁₀(CH₂)₂Si(OC₂H₅)₃, CF₃(CF₂)₉(CH₂)₂Si(OC₂H₅)₃, CF₃(CF₂)₈(CH₂)₂Si(OC₂H₅)₃, CF₃(CF₂)₇(CH₂)₂Si(OC₂H₅)₃, CF₃(CF₂)₆(CH₂)₂Si(OC₂H₅)₃, CF₃(CF₂)₅(CH₂)₂Si(OC₂H₅)₃, CF₃(CF₂)₄(CH₂)₂Si(OC₂H₅)₃, CF₃(CF₂)₃(CH₂)₂Si(OC₂H₅)₃, CF₃(CF₂)₂(CH₂)₂Si(OC₂H₅)₃, CF₃CF₂(CH₂)₂Si(OC₂H₅)₃ and CF₃(CH₂)₂Si(OC₂H₅)₃, triacyloxysilanes containing a fluoroalkyl group such as CF₃(CF₂)₁₁(CH₂)₂Si(OCOCH₃)₃, CF₃(CF₂)₁₀(CH₂)₂Si(OCOCH₃)₃, CF₃(CF₂)₉(CH₂)₂Si(OCOCH₃)₃, CF₃(CF₂)₈(CH₂)₂Si(OCOCH₃)₃, CF₃(CF₂)₇(CH₂)₂Si(OCOCH₃)₃, CF₃(CF₂)₆(CH₂)₂Si(OCOCH₃)₃, CF₃(CF₂)₅(CH₂)₂Si(OCOCH₃)₃, CF₃(CF₂)₄(CH₂)₂Si(OCOCH₃)₃, CF₃(CF₂)₃(CH₂)₂Si(OCOCH₃)₃, CF₃(CF₂)₂(CH₂)₂Si(OCOCH₃)₃, CF₃CF₂(CH₂)₂Si(OCOCH₃)₃ and CF₃(CH₂)₂Si(OCOCH₃)₃, triisocyanate silanes containing a fluoroalkyl group such as CF₃(CF₂)₁₁(CH₂)₂Si(NCO)₃, CF₃(CF₂)₁₀(CH₂)₂Si(NCO)₃, CF₃(CF₂)₉(CH₂)₂Si(NCO)₃, CF₃(CF₂)₈(CH₂)₂Si(NCO)₃, CF₃(CF₂)₇(CH₂)₂Si(NCO)₃, CF₃(CF₂)₆(CH₂)₂Si(NCO)₃, CF₃(CF₂)₅(CH₂)₂Si(NCO)₃, CF₃(CF₂)₄(CH₂)₂Si(NCO)₃, CF₃(CF₂)₃(CH₂)₂Si(NCO)₃, CF₃(CF₂)₂(CH₂)₂Si(NCO)₃, CF₃CF₂(CH₂)₂Si(NCO)₃ and CF₃(CH₂)₂Si(NCO)₃.

Among these, a trialkoxysilane containing a fluoroalkyl group, particularly a fluoroalkyl-trimethoxysilane or a fluoroalkyltriethoxysilane, which has 13 to 22 fluorine atoms is preferably used.

By coating the surface of the flat portion of the substrate of the present invention using the compound illustrated herein alone or in combination with a different substance, a biological substance will be difficult to attach to the flat portion, whereby contamination of a sample of a biological substance into an adjacent recess is not likely to occur even if the recesses are located close to each other.

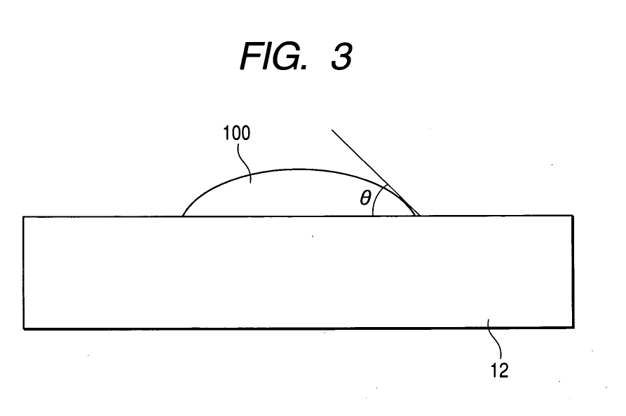

The substrate of the present invention has recesses on the surface thereof in advance, which is different from the substrates disclosed in the foregoing JP-T-9-500568, JP-A-2002-131327, JP-A-2002-307801, JP-A-2002-283530 and JP-A-2003-121442, etc. This recess particularly has a function of retaining liquid. This function of retaining liquid can be evaluated by the contact angle of a liquid on the surface of a solid substrate. The contact angle θ is defined as the angle between the surface of a solid substrate 12 and the tangent line at the point of contact of a liquid droplet 100 with the surface of the substrate as shown in FIG. 3.

In the present invention, the difference in the contact angles for the recess and for the flat portion is made 20 degree or bigger, a substrate for a biochip with excellent quantitativity and reproducibility and with binding sites in a high density can be provided. On the surface of a flat substrate without recesses, a bigger difference in the contact angles is required. Therefore, according to the present invention, the range of choosing a coating material is expanded. The difference in the contact angles is made preferably 50 degree or bigger, more preferably 80 degree or bigger. In this way, a substrate with further more excellent selectivity can be provided.

Incidentally, the maximum contact angle is 180 degree. In this case, a liquid does not wet a substrate at all, and is a droplet in a spherical shape. For the substrate of the present invention, an ideal contact angle on the flat portion which has been given water repellency is 180 degree.

The substrate of the present invention is characterized by having regularly arranged recesses. The shape, height and width of the recess and the density of the recesses may take any suitable form according to a biochip for which the substrate of the present invention is used. Examples of the shape of the recess can include sphere, cone, triangular pyramid, square pyramid, ditch, cylinder, line, Y-branch line and the like.

In the case where the arranged recesses are in a shape of sphere, cone, triangular pyramid, square pyramid, ditch, cylinder or the like, the number of recesses per 1 cm² is set to 4 or more, preferably 100 or more, more preferably 10,000 or more. In addition, in the case of linear recesses, the width of the line is set to 3,000 μm or less, preferably 10 μm or less. In this way, a substrate for a biochip with a structure of fine patterns in a high density can be obtained.

Subsequently, a method of producing the substrate for a biochip of the present invention will be described. Basically, recesses on the surface of the substrate are processed in advance, and then coating films are formed of a material with a desired adhesiveness on the recesses and the flat portion, respectively.

As the method of producing a substrate having regularly arranged recesses, a method of forming a mask pattern by photolithography, electron lithography, proton lithography, X-ray lithography or the like in combination with forming recesses by the laser abrasion method, wet etching method or the like can be exemplified.

As the method of forming a coating film on the surface of the substrate, a wet method or a dry method (vacuum method) can be exemplified.

Examples of the wet method can include the spin coating method, dip coating method, spray coating method, flow coating method, meniscus coating method, gravure printing method, flexographic printing method, nanoimprinting method, soft lithography method, microcontact printing method and the like. In particular, the soft lithography method is a convenient and low-cost method as a means for selectively allowing a solution to adhere to the flat portion of the surface of the substrate having recesses.

Examples of the dry method (vacuum method) can include the vapor deposition method, sputtering method, ion beam method, CVD method, MOCVD method and the like. By combining these methods, a coating film of a specified material can be formed in a specified portion on the surface of the substrate.

Hereunder, specific Examples will be described.

### EXAMPLE 1

On a silica glass substrate (with a thickness of 2 mm and dimensions of 50 mm×50 mm), a Cr film was formed by the sputtering method, and further photoresist was applied thereto by the spin coating method. Then, the photoresist film was exposed to light in a pattern in which 50 openings were regularly arranged vertically and horizontally and a total of 2,500 openings were arranged in a grid, and the exposed portion of the photoresist was developed and removed. Then, by using the photoresist film as a mask, the Cr film was etched, whereby openings were formed.

This Cr film-coated glass substrate with photoresist was washed with ultrapure water (specific resistance value: 18 MΩ·cm), and then etching was carried out with 49% hydrofluoric acid, whereby recesses in a spherical shape were formed. Thereafter, the substrate was washed with ultrapure water, and then the photoresist film was removed with an aqueous solution of NaOH.

In this state, glass of the substrate was exposed on the surface of the recesses, and the flat portion was coated with the Cr film. On the entire surface of the substrate in this state, an Au film was formed by the sputtering method. Then, the Cr mask was stripped off with an aqueous solution of diammonium cerium nitrate, whereby a substrate having an Au film only in the spherical recesses was obtained.

Then, on the flat portion, a water-repellent layer was formed by the soft lithography method as shown in the following.

Polydimethylsiloxane (PDMS) in the shape of a plate with a flat surface and a thickness of about 1 mm was used as a stamper. An alcohol solution of a fluoroalkylsilane hydrolyzed with an acid catalyst and water was added to a container in the shape of a flat dish, and one surface of the stamper was brought into contact with this solution. Then, the stamper was brought into contact with the surface of the foregoing substrate, whereby the solution on the surface of the stamper was transferred on the surface of the substrate. Subsequently, the substrate was dried at room temperature for 24 hours.

When the contact angle of water on the surface of this substrate was measured, it was 110 degree with regard to the surface of the flat portion (Biochip substrate A).

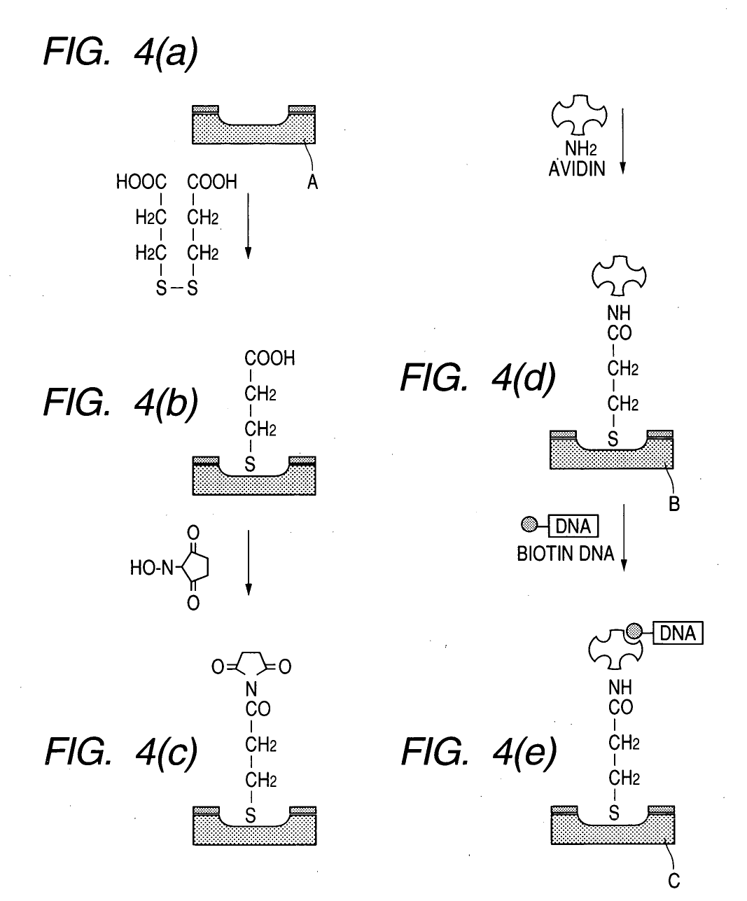

Subsequently, in order to immobilize DNA in the recess of the Biochip substrate A, treatments were carried out by processes as shown in FIG. 4.

Firstly, the Biochip substrate A was dipped for 30 minutes in 3 ml of an aqueous solution of 3,3′-dithiodipropionic acid at a concentration of 1 mM. By doing this, a carboxyl group is introduced on the surface of the Au film ((b) of FIG. 4).

Then, the substrate was dipped in a mixed aqueous solution of N-hydroxysuccinimide and 1-ethyl-3-(3-dimethylaminopropyl) at a concentration of 100 mg/ml, whereby the carboxyl group on the surface of the substrate was reacted with the solution for 30 minutes, and then the substrate was dried. By doing this, an active ester group is introduced on the surface of the Au film ((c) of FIG. 4).

Then, avidin was prepared at a concentration of 0.2 mg/ml with a buffer (pH=8.0, 10 ml of Tris-HCl, 0.2 mol of sodium chloride). In 1 ml of the obtained solution, the substrate was dipped for 1 hour. The substrate was dipped in 1 ml of 1 M ethanol amine aqueous solution for 30 minutes, whereby an unreacted carboxyl group was inactivated. In this way, the Au film in the recess was modified with avidin through a thioether bond ((d) of FIG. 4, Biochip substrate B). This Biochip substrate B is a substrate for a biochip of the present invention with a linker for immobilizing DNA.

By treating this Biochip substrate B as follows, DNA can be immobilized only on the recess of the substrate. Biotinylated DNA was prepared at a concentration of 1 μM with a buffer (pH=8.0, 10 ml of Tris-HCl, 0.2 mol of sodium chloride). In 1 ml of the obtained solution, the Biochip substrate B was dipped at 25° C. for 30 minutes, whereby Biochip substrate C on which biotin-modified DNA was immobilized using avidin as a linker was obtained ((e) of FIG. 4).

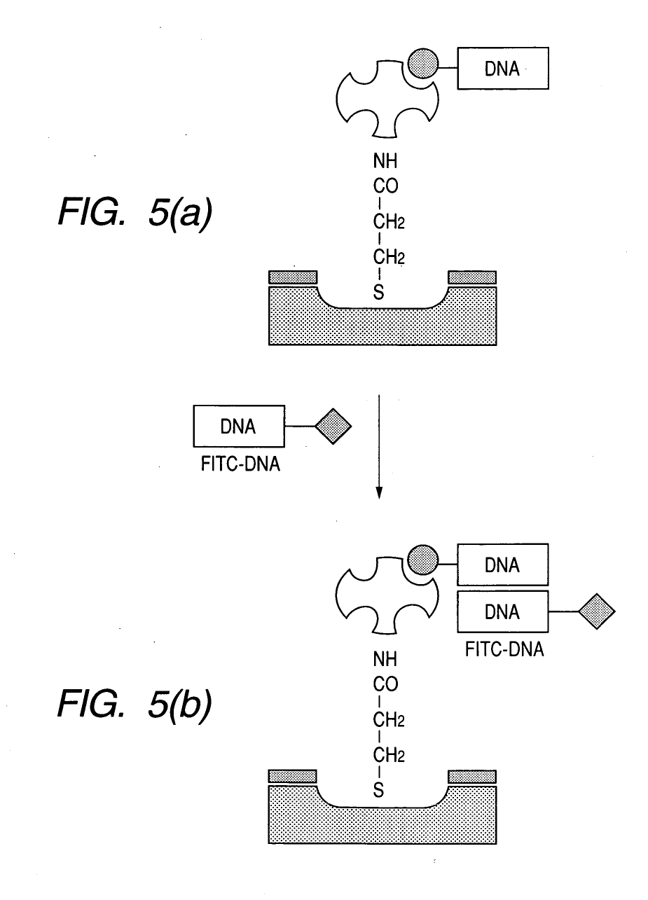

Subsequently, in order to perform observation by enhancing fluorescence intensity, as shown in FIG. 5, DNAs are bound to each other. In 1 ml of a solution in which DNA modified with FITC was diluted with a buffer (pH=7.9, 10 ml of Tris-HCl, 0.2 mol of sodium chloride), the Biochip substrate C was dipped at 60° C. for 30 minutes, whereby DNAs were bound to each other ((b) of FIG. 5). By observing the fluorescence of the bound DNAs with a fluorescence microscope (excitation light at 450 to 490 nm, light absorption at 515 to 565 nm), it was confirmed that DNA was immobilized on the recess of the substrate.

### EXAMPLE 2

In this Example, an alkanethiol was selectively introduced only on the Au film in the recesses of Biochip substrate A produced in the same manner as in Example 1.

An ethanol solution of eicosanethiol [CH₃(CH₂)₁₉SH] (3%) (weight/volume) was prepared. Then, the Biochip substrate A was dipped in this solution and left at room temperature for 3 hours. An alkanethiol did not attach to the water-repellent flat portion, and a film was formed only on the Au film having a high reactivity with a thiol group. Thereafter, by performing the same treatments as in Example 1, a substrate for a biochip on which a linker has been introduced through a thioether bond can be obtained.

### EXAMPLE 3

In this Example, an alkanethiol containing a hydroxyl group was selectively introduced only on the Au film in the recesses of Biochip substrate A produced in the same manner as in Example 1.

An ethanol solution of 11-mercapto-1-undecanol [HO(CH₂)₁₁SH] (3%) (weight/volume) was prepared. Then, the Biochip substrate A was dipped in this solution and left at room temperature for 3 hours. 11-mercapto-1-undecanol did not attach to the water-repellent flat portion, and a film was formed only on the Au film having a high reactivity with a thiol group. Thereafter, by performing the same treatments as in Example 1, a substrate for a biochip on which a linker has been introduced through a thioether bond can be obtained.

### EXAMPLE 4

In this Example, an alkanethiol containing a carboxyl group was selectively introduced only on the Au film in the recesses of Biochip substrate A produced in the same manner as in Example 1.

An ethanol solution of 16-mercaptohexadecanoic acid [HOOC(CH₂)₁₅SH] (3%) (weight/volume) was prepared. Then, the Biochip substrate A was dipped in this solution and left at room temperature for 3 hours. 16-mercaptohexadecanoic acid did not attach to the water-repellent flat portion, and a film was formed only on the Au film having a high reactivity with a thiol group. Thereafter, by performing the same treatments as in Example 1, a substrate for a biochip on which a linker has been introduced through a thioether bond can be obtained.

### EXAMPLE 5

In this Example, an alkanethiol containing an amino group was selectively introduced only on the Au film in the recesses of Biochip substrate A produced in the same manner as in Example 1.

An ethanol solution of 11-amino-1-undecanethiol [H₂N(CH₂)₁₁SH] (3%) (weight/volume) was prepared. Then, the Biochip substrate A was dipped in this solution and left at room temperature for 3 hours. 11-amino-1-undecanethiol did not attach to the water-repellent flat portion, and a film was formed only on the Au film having a high reactivity with a thiol group. Thereafter, by performing the same treatments as in Example 1, a substrate for a biochip on which a linker has been introduced through a thioether bond can be obtained.

### EXAMPLE 6

On a silica glass substrate (with a thickness of 2 mm and dimensions of 50 mm×50 mm), a Cr film was formed by the sputtering method, and further photoresist was applied thereto by the spin coating method. Then, the photoresist film was exposed to light in a pattern in which 50 openings were arranged vertically and horizontally and a total of 2,500 openings were arranged in a grid, and the exposed portion of the photoresist was developed and removed. Then, by using the photoresist film as a mask, the Cr film was etched, whereby openings were formed.

This Cr film-coated glass substrate with photoresist was washed with ultrapure water (specific resistance value: 18 MΩ·cm), and then etching was carried out with 49% hydrofluoric acid, whereby recesses in a spherical shape were formed. Thereafter, the substrate was washed with ultrapure water, and then the photoresist film was removed with an aqueous solution of NaOH. Further, by using an aqueous solution of diammonium cerium nitrate, the Cr mask was stripped off.

Then, on the flat portion, a water-repellent layer was formed by the soft lithography method as shown in the following.

Polydimethylsiloxane (PDMS) in the shape of a plate with a flat surface and a thickness of about 1 mm was used as a stamper. An alcohol solution of a fluoroalkylsilane hydrolyzed with an acid catalyst and water was added to a container in the shape of a flat dish, and one surface of the stamper was brought into contact with this solution. Then, the stamper was brought into contact with the surface of the foregoing substrate, whereby the solution on the surface of the stamper was transferred on the surface of the substrate. Subsequently, the substrate was dried at room temperature for 24 hours.

When the contact angle of water on the surface of this substrate was measured, it was 110 degree with regard to the surface of the flat portion.

Subsequently, a portion corresponding to the flat portion of this glass substrate was shielded, and a mask made of glass having openings only at the sites corresponding to the recesses was prepared. The positions of the openings of this mask and the recesses of the substrate were fitted and attached together. Then, an Ag film was formed only in the recesses by the sputtering method. By using this substrate instead of the Biochip substrate A in Example 1, a thiol compound was formed into a film selectively only on the Ag film in the recesses. Thereafter, by performing the same treatments as in Example 1, a substrate for a biochip on which a linker has been introduced through a thioether bond can be obtained.

### EXAMPLE 7

A substrate for a biochip in which a thiol compound was formed into a film selectively only on the Cu film in the recesses was obtained in the same manner as in Example 6 except for forming a Cu film instead of an Ag film.

### EXAMPLE 8

A substrate for a biochip in which a thiol compound was formed into a film selectively only on the Pd film in the recesses was obtained in the same manner as in Example 6 except for forming a Pd film instead of an Ag film.

In a substrate for a biochip of the present invention, a small amount of a specific substance can be stably attached or retained in a recess, and contamination into an adjacent recess can be prevented. In addition, the variation in the amount of the attached substance can be reduced, and the repetitive reproducibility can be improved, thus a substrate for a biochip having an excellent function of attachment or retention can be provided.

The entire disclosure of each and every foreign patent application from which the benefit of foreign priority has been claimed in the present application is incorporated herein by reference, as if fully set forth.

1. A substrate having a plurality of recesses,
wherein each of the plurality of recesses has a surface,
wherein at least part of the surface is coated with a metal film comprising at least one element selected from Au, Ag, Cu and Pd.

2. The substrate according to claim 1,
wherein the plurality of recesses are regularly arranged.

3. The substrate according to claim 1,
wherein a linker for immobilizing a biological substance is bound to the metal film.

4. The substrate according to claim 3,
wherein the liker has a thioether bond bound to the metal film.

5. The substrate according to claim 1,
wherein a surface of the substrate other than the at least part of the surface is coated with a water-repellent film.

6. A biochip substrate comprising:
a substrate having at least one recess; and
a metal film formed on the at least one recess,
wherein the metal film comprises at least one element selected from Au, Ag, Cu and Pd.

7. The biochip substrate according to claim 6,
wherein the metal film covers the at least one recess entirely.

8. The biochip substrate according to claim 6,
wherein the metal film is coated on a bottom portion of the at least one recess.

9. The biochip substrate according to claim 6,
wherein the at least one recess is regularly arranged.

10. The biochip substrate according to claim 6, which further comprises a linker for immobilizing a biological substance,
wherein the linker is bound to the metal film.

11. The biochip substrate according to claim 10,
wherein the liker has a thioether bond bound to the metal film.

12. The biochip substrate according to claim 6, which further comprises a water-repellent film covering a surface of the biochip other than a surface of the metal film.

13. The biochip substrate according to claim 12, wherein the water-repellent film further covers a part of a surface of the at least one recess.
